(19) United States
(12) Patent Application Publication
(10) Pub. No.: US 2023/0060633 A1
(43) Pub. Date: Mar. 2, 2023

(54) Testing System For Pathogens and Analytes

(71) Applicant: Wylo Biosciences LLC, Shelburne, VT (US)

(72) Inventors: Ethan Alden-Danforth, Shelburne, VT (US); Joshua Arvin, Needham, MA (US); Anna Jane Brown, Wethersfield, CT (US); Hunter Duger, Auburn, NY (US); Joshua Miller, Wayne, NJ (US); Calvin Pease, Morrisville, VT (US)

(73) Assignee: Wylo Biosciences LLC, Shelburne, VT (US)

(21) Appl. No.: 17/823,144

(22) Filed: Aug. 30, 2022

Related U.S. Application Data
(60) Provisional application No. 63/238,860, filed on Aug. 31, 2021.
(60) Provisional application No. 63/371,803, filed on Aug. 18, 2022.

(51) Int. Cl. G01N 33/52; G01N 21/78; B01L 3/00; G01N 33/569

(52) U.S. Cl. CPC G01N 33/521; G01N 21/78; B01L 3/5023; G01N 33/569; G01N 2001/2866

(57) ABSTRACT

A device to detect pathogens and/or analytes in a test sample is designed to include a core, which may have first and second chambers. The first chamber is designed to accept a combination of a test specimen and a buffer liquid. A piston is designed to slideably engage into the first chamber of the core, wherein the end of the piston may engage with the bottom of the first chamber and be capable of grinding the test specimen. A plunger is designed to slideably engage with the second chamber of the core, and is in fluid communication with the first chamber. An assay section is attached to the core, and is in fluid communication with the second chamber. The assay device is designed to include a test strip that is capable of detecting and indicating the presence of pathogens and/or analytes in the combination of the test sample and buffer liquid.

### CROSS-REFERENCE TO RELATED APPLICATION

This application claims the benefit of provisional application No. 63/238,860 filed Aug. 31, 2021; and provisional application No. 63/371,803 filed Aug. 18, 2022; the entire disclosures of the parent applications are incorporated herein by reference.

### FEDERALLY-SPONSORED RESEARCH

None

### BACKGROUND

The present disclosure relates to a testing system for pathogens.

### BRIEF DESCRIPTION

In general, a testing device and related method is designed with a piston, which may include a piston chamber, a first retention portion, an annular lower portion, a pierceable layer spanning the annular lower portion's center, a textured surface at a bottom end of the piston, the piston chamber capable of retaining a fluid. A testing device may include a core, with a first core chamber, a second core chamber, a second retention portion, at least one lower drain hole, an annular grinding surface, an assay opening, and a piercing element. A testing device may include a plunger, with at least one hole plug protruding from a top portion of the plunger, a circumferential sealing element, and a retaining element; and may also include an assay section, with a test strip, an indicator window, and a plunger retaining element.

The piston may be operatively connectable to the core such that pressing the piston into the first core chamber causes the piercing element to pierce the pierceable layer, with the piston capable of rotating inside of the core chamber. The plunger may be operatively connectable to the core such that extending the plunger relative to the core removes the at least one hole plug from the at least one drain hole. The first core chamber may be capable of receiving a test specimen prior to the piston being inserted into the core, allowing the test specimen to be ground between the textured surface and the grinding surface.

The textured surface may contact the grinding surface when the piston is pressed fully into the core. The buffer liquid contained in the piston chamber may flow into the core chamber when the pierceable layer is pierced. Extending the plunger relative to the core may allow a combination of the buffer liquid and the test specimen to flow through the at least one hole from the first core chamber to the second core chamber. A combination of the buffer liquid and the test specimen may pass through the assay opening to make fluid contact with the test strip. The test strip may be configured to detect and indicate the presence of pathogens in a combination of the buffer liquid and the test specimen, where the indication may be visual and viewable on the test strip through the indicator window. Pathogens detected may be from a test specimen that is a tick.

The piercing element may include a sculpted surface. The plunger may include a base which allows the testing device to stand in a vertical position. The textured surface may be contained on a texture plate, the texture plate affixable to the annular lower portion of the piston. The pierceable layer may be positioned between the texture plate and the annular lower portion of the piston. Rotation of the piston, with the piston fully compressed into the core, may cause the test specimen to be ground and biological contents to be released into the buffer liquid. The piston's first retention portion may engage with the core's second retention portion. The assay's plunger retaining element may engage with the core's retaining element to prevent the plunger from being removed from the core.

There is also a testing device that may include a vial with a first closed end and an opposing open end, the open end capable of receiving a test specimen and a buffer liquid; a sliding member with a cap end and an opposing cylindrical end, the two ends connected by a rod, the cap end containing a slot, with a filter positioned at the end of the slot facing the cylindrical end. The sliding member may be configured to be slidable inside of the vial and capable of crushing the test specimen against the first closed end. In conjunction with the above, a lateral flow assay device may be used, with an elongated tip, the tip configured to enter the filter through the slot, the lateral flow assay device configured to detect and indicate the presence of pathogens in a combination of the buffer liquid and the test specimen.

### BRIEF DESCRIPTION OF THE FIGURES

FIG. 1 shows a perspective view of vial and a sliding member assembly;

FIG. 2 shows a front perspective view of a vial;

FIG. 3 shows a perspective view of an assay device;

FIG. 4 shows a perspective view of a removal device;

FIG. 5 shows a perspective view of a test system;

FIG. 6 shows an exploded perspective view of a test system including a test specimen;

FIG. 7 shows an exploded right view of a test system;

FIG. 8 shows an exploded perspective view a core, test paper, and assay cover, as part of a test system;

FIG. 9 shows an exploded perspective view of a piston and texture plate, as part of a test system;

FIG. 10 shows an exploded perspective view of a plunger and sealing element, as part of a test system;

FIG. 11 shows a side cross-sectional view of a core, as part of a test system.

### DETAILED DESCRIPTION

The present system relates to a testing system for pathogens or analytes that may be present in a test specimen. In one embodiment, the device may be used to determine whether test specimens such as ticks contain pathogens that can cause diseases including but not limited to Anaplasmosis, Babesiosis, Ehrlichiosis, Heartland Virus, Lyme Disease, Powassan Virus, Rickettsiosis, Rocky Mountain Spotted Fever, Southern Tick Associated Rash Illness, Tick-Borne Relapsing Fever, and Tularemia. In another embodiment, the device may be used to determine whether other biological test specimens contain analytes such as pharmaceutical drugs, allergens, narcotics, non-microbial contaminants, toxins, ions, or other small organic or non-organic molecules.

The device may also be used for the preparation and testing of other test specimen and biological matter types, including but not limited to: various insect types, fecal samples, meat, fish, vegetables, legumes or fruits, grain, dirt, minerals, water, bodily tissue (as in a biopsy sample), renal calculus, hair, blood, serum, saliva, semen, synovial fluid, cerebrospinal fluid, lotion, leaf, grass, vegetation, flower, root, or stem, any which may carry a variety of pathogen types. Thus, reference to tick or insect may be interchanged with at least any of the above types for purposes of this disclosure.

Through this disclosure buffer, or buffer liquid, may refer to a common solution as known in the art. It may include buffering agents to stabilize pH, lysing agents, surfactants, and other ingredients as appropriate for an application. Embodiments in the disclosure may be manufactured by any means in the art, such as but not limited to plastic injection molding, machining, 3-D printing, etc.

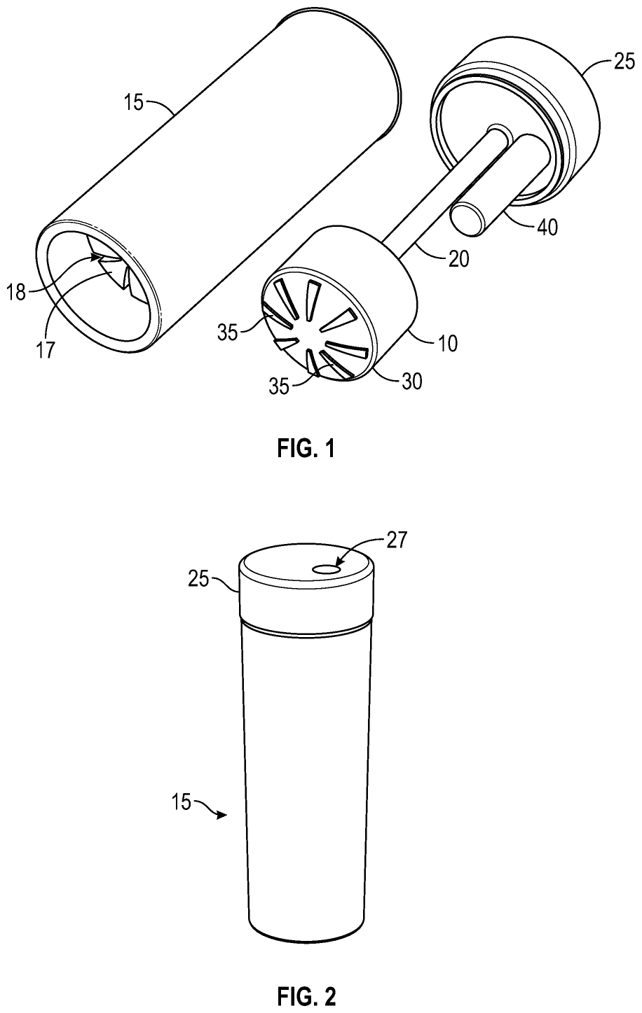

Referring to FIG. 1, in one embodiment there may be a vial 15, which includes a closed end bottom 17, with the opposing end open. Sliding member 10 includes two ends: at one end is cap 25, and at the other is second end 30, with rod 20 connecting the two ends. A plurality of ridges 35 may be present on the bottom of second end 30. Filter 40 may be affixed to the underside of cap 25. As seen in FIG. 2, there may be a slot 27 that penetrates the depth of cap 25. Slot 27 is positioned to align with filter 40. Sliding member 10 is capable of sliding inside of vial 15, with end 30 abutting bottom 17, when the sliding member is fully inserted into the vial. Bottom 17 may include ridges 18, which creates a coarse surface inside of vial 15 to aid in the crushing of an insect.

The inside of the closed end of vial 15 may be conical-shaped. Similarly, second end 30 with ridges 35 may have a similar curved surface to accelerate the process of grinding a test specimen when cap 25 is inserted, closed, and moved back-and-forth and/or rotated in a circular motion. Cap 25 may include a closure that locks into the top of vial 15 to ensure no spillage if the vial-sliding member assembly is in a non-vertical position.

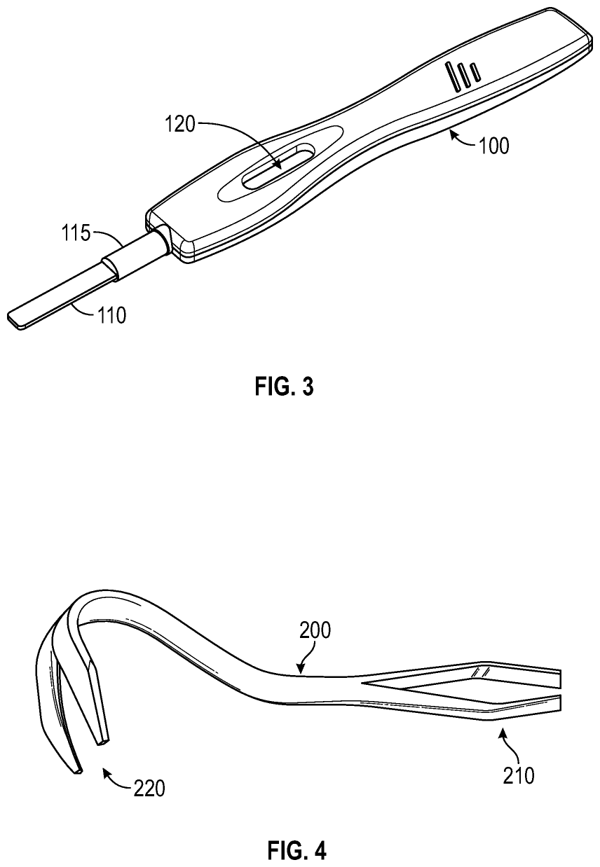

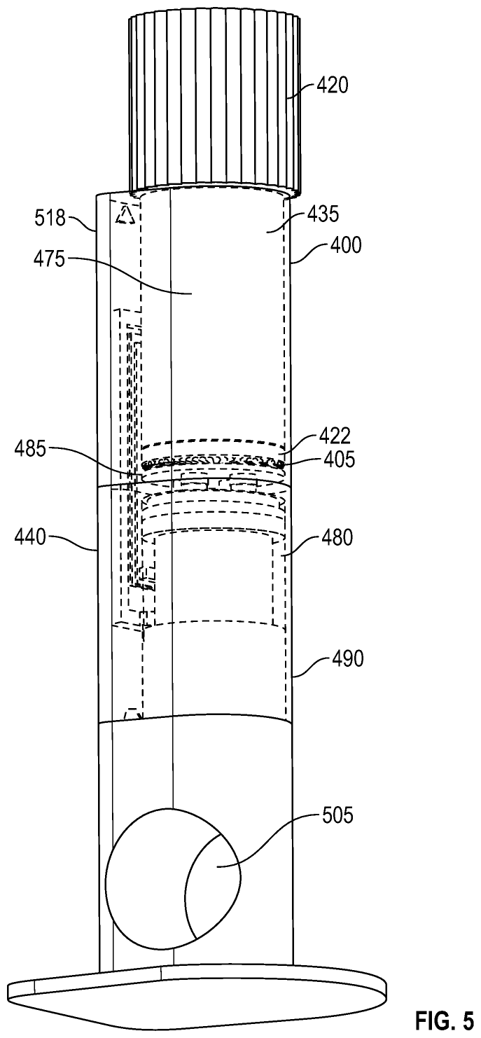

FIG. 3 shows a lateral flow assay device. This includes body 100 with an elongated tip. The elongated tip may comprise connector 115, and paper strip 110. Window 120 may be present to display test results, as will be explained. FIG. 4 shows remover 200, which may have two ends. At one end may be tweezer arms 210, and the other end may include claw 220. Claw 220 may have a curved fulcrum profile.

A first step in testing, such as testing a tick for various pathogens, involves crushing and sample preparation. A tick or insect is placed into vial 15, and approximately 1 mL of buffer liquid is added into vial 15. The buffer liquid may be provided in ampoule or other suitable container. Sliding member 10 is inserted into vial 15, with cap 25 inserted into vial 15 and closed securely. A user then holds vial 15 upright and rotates (or uses and up-and-down motion) cap 10 a plurality of times, thereby crushing the insect and releasing its inner biological contents and any bacteria potentially within.

The next testing step involves placing tip 110 of the lateral flow assay device into slot 27. The assembly of vial 15 and sliding member 10 is then turned 90 degrees onto its side to allow the crushed insect liquid to flow onto filter 40, thereby making contact with paper tip 110. Insect bacteria is carried by the buffer liquid, with any solid matter from the insect not capable of passing through filter 40 and reaching paper tip 110. The assay device includes, internally, suitable analytic chemicals and/or mechanisms to determine the presence of pathogens of interest absorbed by paper tip 110. Different analytics are used to detect pathogens of interest for specific applications. The user then waits a predetermined amount of time until a result appears in the viewing window 120, either positive or negative, indicating presence or absence of a pathogen.

If an insect to be tested is attached to the body of a creature (human, dog, etc.) remover 200 may be used to help remove the insect. Claw 20 may be wedged between an insect and a creature's skin, with the insect pried away. Tweeze ends 210 may also be used.

FIG. 5 shows a perspective view of a different embodiment of a test system. Piston 400 may include a piston chamber 435. Piston chamber 400 is capable of retaining a fluid, such a buffer liquid. A pierceable layer 410 may span the center of the annular lower portion 425. Thus piston chamber 435 may be filled with fluid through the center of annular lower portion 425 (such as when the piston in is an inverted position to that shown), with pierceable layer 410 placed across the center opening of annular lower portion 425, thus sealing the piston chamber 435. Pierceable layer 410 may be in the form of an adhesive sticker or other suitable type. A gripping portion 420 may be present on piston 400.

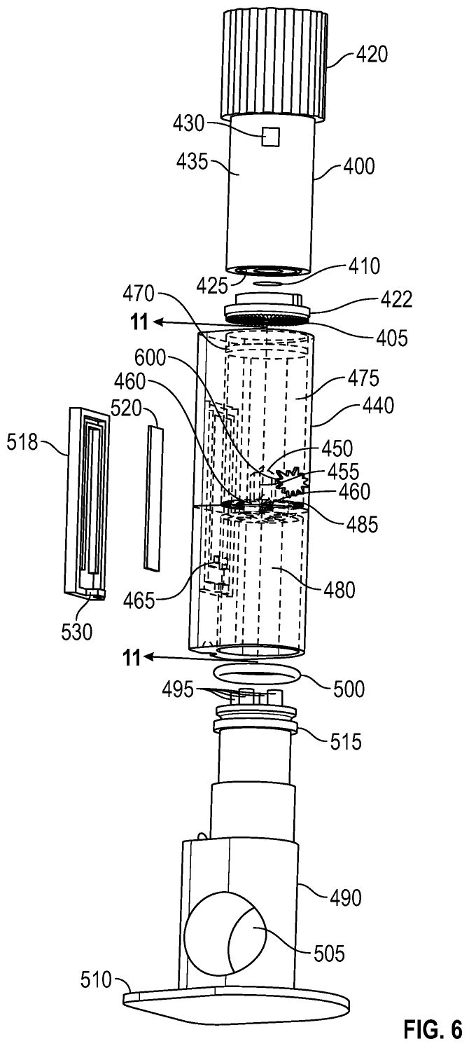

Piston 400 may be configured with a textured surface 405 its bottom end. The textured surface may be part of piston 400 itself, or may be a separate texture plate 422 that can be affixed to piston 400. Texture plate 422 may be affixed to piston 400 after the pierceable layer 410 is applied to the annular lower portion 425. A separate texture plate 422 can be seen in FIG. 6 and FIG. 9.

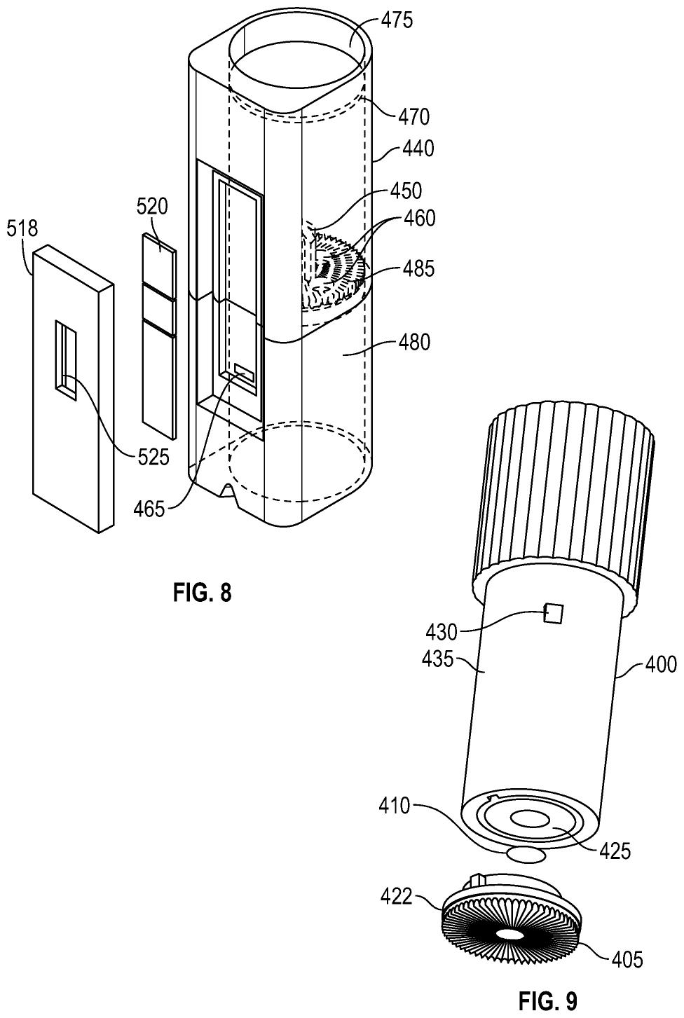

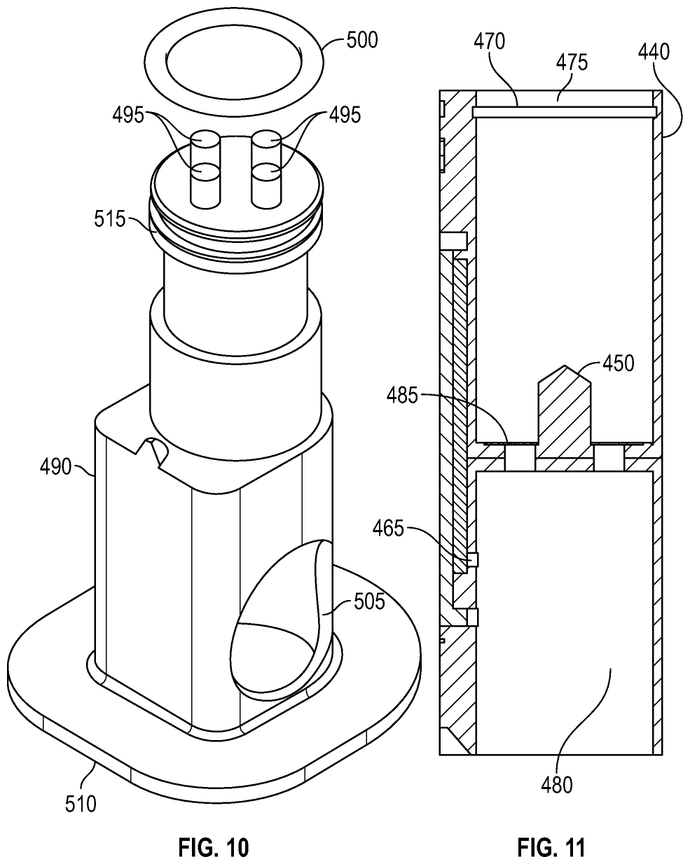

The exploded view of FIG. 6 shows that piston 400 may be configured to slideably engage with a first core chamber 475 of core 440. The lower portion of first core chamber 475 is configured to include an annular grinding surface 485, best seen in FIG. 8. A piercing element 450 is configured in the center of annular grinding surface 485, extending upwards. Piercing element 450 may be configured to include sculpted surface 455 which may aid in the grinding of a test specimen 600 (test specimen 600 not shown in other views, for clarity), as will be detailed. At least one lower drain hole 460 is present on annular grinding surface 485—with four holes shown in the various figs herein.

FIG. 6 also shows first retention portion 430 (on piston 400) with second retention portion 470 (on core 440). First retention portion 430 may be wedged-shaped, using a ramped profile that allows 430 to snap into second retention portion 470, when piston 400 is inserted into core 440. Second retention portion 470 may be in the form of a circumferential groove on the inside of core 440. This allows piston 400 to be inserted into core 440 one-time, with the piston not readily removable once the protruding first retention portion 430 engages into the groove of second retention portion 470.

Core 440 may be configured with a second core chamber 480, positioned below first core chamber 475. The two chambers 475 and 480 are in fluid communication with each other through at least one lower drain hole 460. Second core chamber 480 is configured with an assay opening 465, which allows fluid communication with assay section 518, including test strip 520. Test strip 520 may be of any suitable type known in the art. It may be made of nitrocellulose paper, or other suitable material.

The exploded view of FIG. 6 also shows that plunger 490 may be configured to slideably engage with a second core chamber 480 of core 440. Plunger 490 may be configured to include at least one hole plug 495 protruding from a top portion. The number of plugs 495 matches the number of drain holes 460, with plugs 495 sealing holes 460 when plunger 490 is fully inserted into core 440. A circumferential sealing element 500 sits in a grove on the upper portion of plunger 490, to create a seal inside second core chamber 480. Element 500 may be in the form of an O-ring, made from rubber, plastic, or other suitable material. Plunger 490 may also include gripping hole 505, and base 510—the latter allowing the overall device to stand in a vertical orientation.

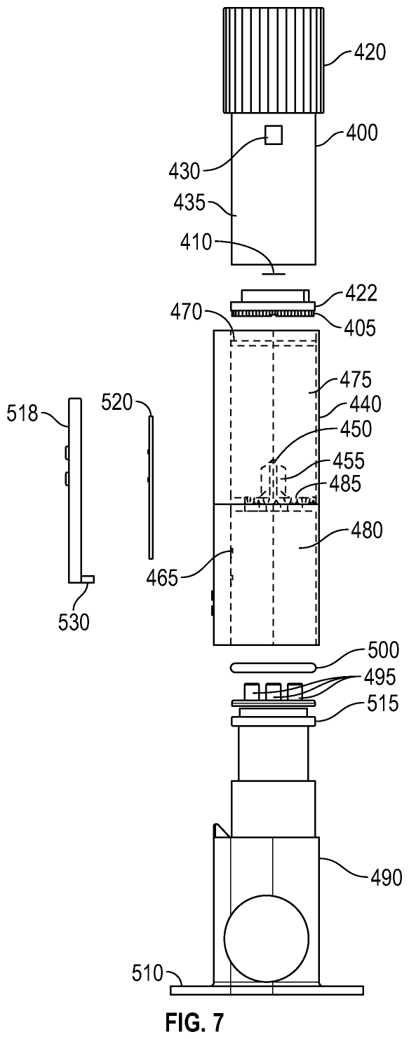

Referencing FIG. 8, assay section 518 may be configured to include test strip 520, and indicator window 525. Assay section 518 is affixed to the side of core 440. Assay opening 465 (on core 440) allows test strip 520 to be in fluid communication with second core chamber 480. FIG. 7 shows plunger retaining element 515 (on plunger 490), and retaining element 530 (on assay 518)—these elements work together to limit travel and prevent plunger 490 from being fully removed from core 440, with retaining element 530 not allowing plunger retaining element 515 to travel past. In manufacturing, plunger 490 is inserted into core 440 prior to assay section 518 being affixed to core 440.

The system may be designed to function in a number of different ways, including the following. A user begins with the system as configured from manufacturing with plunger 490 fully seated into core 440, assay section 518 affixed to core 440, with piston 400 separated from core 440. Piston chamber 435 contains an appropriate amount of buffer liquid, which was installed during manufacturing, with pierceable layer 410 sealing the buffer liquid in piston chamber 435.

A user places a test specimen such as 600 into first core chamber 475, wherein it rests on annular grinding surface 485, which may include a textured surface. Piston 490 is then fully inserted to first core chamber 475. Such insertion causes piercing element 450 to pierce pierceable layer 410, causing the buffer liquid to be released into first core chamber 475. Sculpted surface 455 on piercing element 450 in part allows the buffer liquid to flow into the chamber.

A user imparts motion to piston 400, while holding the core in place. Motion may be rotations of piston 400 and/or an up-and-down motion—both serve to grind the test specimen between textured surface 405 and annular grinding surface 485. The dimension of first retention portion 430 relative to the groove on second retention portion 470 may be such that a small amount of up-and-down motion is possible, with 430 movable within 470. After the test specimen is ground as desired, the inner biological contents of the test specimen (such as insect internal matter and pathogens) will form a combination with the buffer liquid.

The user then extends plunger 490 relative to core 440, which removes the at least one hole plug 495 from the at least one lower drain hole 460, causing the combination to drain into second core chamber 480. The combination may then travel through assay opening 465. This allows the combination to be in fluid contact with test strip 520. Retaining element 515 in combination with plunger retaining element 530 prevents plunger 490 from being over-extended and removed from core 440.

The test strip includes, internally, suitable analytic chemicals and/or mechanisms to determine the presence of pathogens of interest absorbed by the test strip. Different analytics are used to detect pathogens of interest for specific applications. The user then waits a predetermined amount of time until a result appears in the indicator window 525, either positive or negative, indicating presence or absence of a pathogen.

Although the present system has been described with respect to one or more embodiments, it will be understood that other embodiments of the present system may be made without departing from the spirit and scope of the present system. Hence, the present system is deemed limited only by claims and the reasonable interpretation thereof.

We claim:

1. A testing device, comprising:
a piston, with a piston chamber, an annular lower portion, a pierceable layer spanning the annular lower portion's center, a textured surface at a bottom end of the piston, the piston chamber capable of retaining a fluid;
a core, with a first core chamber, a second core chamber, at least one lower drain hole, an annular grinding surface, an assay opening, and a piercing element;
a plunger, with at least one hole plug protruding from a top portion of the plunger, and a circumferential sealing element;
an assay section, with a test strip, and an indicator window;
the piston operatively connectable to the core such that pressing the piston into the first core chamber causes the piercing element to pierce the pierceable layer, with the piston capable of rotating inside of the core chamber;
the plunger operatively connectable to the core such that extending the plunger relative to the core removes the at least one hole plug from the at least one lower drain hole, and;
in which the first core chamber is capable of receiving a test specimen prior to the piston being inserted into the core, to allow the test specimen to be ground between the textured surface and the annular grinding surface.

2. The device of claim 1, in which the textured surface contacts the grinding surface when the piston is pressed fully into the core.

3. The device of claim 1, in which a buffer liquid contained in the piston chamber flows into the core chamber when the pierceable layer is pierced.

4. The device of claim 3, in which extending the plunger relative to the core allows a combination of the buffer liquid and the test specimen to flow through the at least one hole from the first core chamber to the second core chamber.

5. The device of claim 4, in which a combination of the buffer liquid and the test specimen passes through the assay opening to make fluid contact with the test strip.

6. The device of claim 5, in which the test strip is configured to detect and indicate the presence of pathogens in a combination of the buffer liquid and the test specimen.

7. The device of claim 6, in which the indication is visual and viewable on the test strip through the indicator window.

8. The device of claim 6, in which the test specimen is a tick.

9. The device of claim 1, in which the piercing element includes a sculpted surface.

10. The device of claim 1, in which the plunger includes a base which allows the testing device to stand in a vertical position.

11. The device of claim 1, in which the textured surface is contained on a texture plate, the texture plate affixable to the annular lower portion of the piston.

12. The device of claim 11, in which the pierceable layer is positioned between the texture plate and the annular lower portion of the piston.

13. The device of claim 1, in which rotation of the piston, with the piston fully compressed into the core, causes the test specimen to be ground and biological matter to be released into the buffer liquid to form a combination.

14. The device of claim 1, in which the piston includes a gripping surface, and the plunger includes a gripping hole.

15. The device of claim 1, in which a plunger retaining element on the assay engages with a retaining element on the core to prevent the plunger from being removed from the core.

16. A testing method, comprising:
configuring a piston with a piston chamber, an annular lower portion, a pierceable layer spanning the center of the annual lower portion, a textured surface at a bottom end of the piston, the piston chamber capable of retaining a fluid;
configuring a core, with a first core chamber, a second core chamber, at least one lower drain hole, an annular grinding surface, an assay opening, and a piercing element;
configuring a plunger, with at least one hole plug protruding from a top portion of the plunger, and a circumferential sealing element;
configuring an assay section, with a test strip, and an indicator window;
operatively connecting the piston to the core such that pressing the piston into the core causes the piercing element to pierce the pierceable layer, with the piston capable of rotating inside of the core chamber;
operatively connecting the plunger to the core such that extending the plunger relative to the core removes the at least one hole plug from the at least one drain hole;
configuring the first core chamber to be capable of receiving a test specimen prior to the piston being inserted into the core, allowing the test specimen to be ground between the textured surface and the annular grinding surface;
allowing a buffer liquid contained in the piston chamber to flow into the first core chamber when the pierceable layer is pierced;
grinding the test specimen between the textured surface and the annular grinding surface to create a combination of the buffer liquid and the test specimen in the first core chamber;
extending the plunger relative to the core to allow the combination of the buffer liquid and the test specimen to flow through the at least one hole from the first core chamber to the second core chamber;
allowing the combination of the buffer liquid and the test specimen to pass through the assay opening to make fluid contact with the test strip, and;
configuring the test strip to detect and indicate the presence of a pathogen in the combination of the buffer liquid and the test specimen.

17. The method of claim 16, in which the piercing element includes a sculpted surface.

18. The method of claim 16, in which the plunger includes a base which allows the testing device to stand in a vertical position.

19. The method of claim 16, in which the test specimen is a tick.

20. A testing device, comprising:
a vial with a first closed end and an opposing open end, the open end capable of receiving a test specimen and a buffer liquid;
a sliding member with a cap end and an opposing cylindrical end, the two ends connected by a rod, the cap end containing a slot, with a filter positioned at an end of the slot facing the cylindrical end;
the sliding member configured to be slidable inside of the vial and capable of crushing the test specimen against the first closed end;
a lateral flow assay device with an elongated tip, the tip configured to enter the filter through the slot, the lateral flow assay device configured to detect and indicate the presence of a pathogen in a combination of the buffer liquid and the test specimen.
